(19) United States
(12) Patent Application Publication
(10) Pub. No.: US 2009/0098014 A1
(43) Pub. Date: Apr. 16, 2009

(54) Structure and Method of Air Purification

(76) Inventor: Derek Elden Longstaff, Phoenix, AZ (US)

Correspondence Address: BOOTH UDALL, PLC, 1155 W. Rio Salado Pkwy., Suite 101, Tempe, AZ, 85281, US

(21) Appl. No.: 11/871,948

(22) Filed: Oct. 12, 2007

(51) Int. Cl. A61L 9/20

(52) U.S. Cl. 422/4; 422/121

(57) ABSTRACT

In an example method of purifying air, steps may include: using a motor driven fan wherein the motor has at least two speeds; using an indoor air handling system with the motor driven fan and with a baffle that has at least an open and a closed position; installing a bypass for air to flow through a constricted space around the baffle, so that air can flow past the baffle when the baffle is in the closed position and through the bypass and back into the air handling system; and disinfecting air in the bypass using a UV lamp and a photocatalyst.

### BACKGROUND

1. Field of the Invention

The present invention relates to the field of indoor air purification for purifying air in an inhabited area.

2. Discussion of Related Art

Indoor air pollution is a common problem, as many people spend the vast majority of their time indoors. Indoor air quality is affected by airborne micro-organisms including mold, bacteria, viruses and fungi. Indoor air also at times is polluted by chemical vapors or undesirable chemical compounds, some examples are vapors resulting from cleaning processes, and gases from curing paint. Homes and offices can contain viruses in the air which spread disease through the air handling system in the home or office. Some systems have been developed where UV lamps are placed inside an air handling system in an effort to disinfect the air. The prior systems place the UV lamps inside the conventional air flow of air handling systems, where there is a high rate of air flow. These systems generally encounter the problem that the air is not in the presence of UV lamps long enough to have a very high rate of killing or neutralizing the harmful substances in the air. Some systems have sought to address this problem by placing many more UV lamps in the path of air flow, which greatly increases the cost of the system. There is a need for a system and method of air purification which disinfects air to reduce the living micro-organisms in the air in an efficient manner.

### SUMMARY

In an example embodiment, the system disclosed allows air in an air handling system to bypass into a UV air purification system. Several example embodiments are discussed.

### FIRST EXAMPLE

In one example of a method of purifying air, steps may include forcing air from within an inhabited space into an air handling system using at least one motor driven fan; filtering air using a particulate filter; exposing the forced air to a first UV radiation source and to a second UV radiation source; and returning the air to the inhabited space.

The first UV radiation source may be in a mirrored constricted tubular space with the first UV radiation source in the mirrored constricted tubular space. The mirrored constricted tubular space may have a mirrored surface on an inner wall of the tubular space. The mirrored surface may be capable of reflecting ultraviolet radiation. The mirrored constricted tubular space may constrict the forced air so that the forced air passes within about 3 inches or less from the first UV radiation source.

The second UV radiation source may be a black light and may be in a photocatalyst constricted tubular space with the second UV radiation source in the photocatalyst constricted tubular space. The second tubular space may have a coating of titanium dioxide on a surface of an inner wall of the photocatalyst constricted tubular space. The photocatalyst constricted tubular space may constrict the forced air so that the forced air passes within 3 inches or less from the second UV radiation source.

In alternative embodiments of the present example, forced air may flow first through the mirrored constricted tubular space, and then through the photocatalyst constricted tubular space. The forced air may also flow first through the photocatalyst constricted tubular space and then through the mirrored constricted tubular space. The forced air may also flow in parallel through the mirrored constricted tubular space and the photocatalyst constricted tubular space.

In other alternative embodiments of the present example, the mirrored constricted tubular space may be a mirrored round cylinder and the photocatalyst constricted tubular space may be a second round cylinder. The mirrored constricted tubular space may also be a mirrored square tube and the photocatalyst constricted tubular space may be a second square tube.

In another alternative embodiment of the present example the step of forcing air from within an inhabited space into an air handling system using at least one motor driven fan, may include forcing air at a normal flow rate for heating or cooling air along a first path, and forcing air at a slow flow rate along a second path.

### SECOND EXAMPLE

In an example of a system for purifying air, elements may include a motor driven fan; a particulate filter; a first UV radiation source and a second UV radiation source.

The first UV radiation source of the present example may be in a mirrored constricted tubular space with the first UV radiation source approximately in the center of the mirrored constricted tubular space. The mirrored constricted tubular space may have a mirrored surface on an inner wall of the tubular space. The mirrored surface may be capable of reflecting ultraviolet radiation. The mirrored constricted tubular space may constrict the forced air so that the forced air passes within about 6 inches or less from the first UV radiation source.

The second UV radiation source of the present example may be a black light and may be in a photocatalyst constricted tubular space with the second UV radiation source approximately in the center of the photocatalyst constricted tubular space. The second tubular space having a coating of titanium dioxide on a surface of an inner wall of the photocatalyst constricted tubular space, the photocatalyst constricted tubular space constricting the forced air so that the forced air passes within 6 inches or less from the second UV radiation source;

The motor driven fan of the present example may cause air from an inhabited space to be circulated first through a particulate filter, and then through the mirrored constricted tubular space, and through the photocatalyst constricted tubular space. The motor driven fan may cause the air to be driven through a heating or cooling system and returned to an inhabited space.

In an alternate embodiment of the present example, the motor driven fan may be configured to operate in a low speed and a high speed. The heating or cooling system may have a baffle which is open when the motor driven fan is operating at a high speed, and is closed when the motor driven fan is operating at a low speed. The mirrored constricted tubular space and the photocatalyst constricted tubular space may be connected as to bypass the baffle so that when the baffle is closed, air will flow primarily through the mirrored constricted tubular space and the photocatalyst constricted tubular space at a low speed; and when the baffle is open the air will flow past the open baffle, and through the mirrored constricted tubular space and the photocatalyst constricted tubular space.

In an alternative embodiment of the present example, the air may flow first through the mirrored constricted tubular space and then through the photocatalyst constricted tubular space. The air may also flow in parallel through the mirrored constricted tubular space and the photocatalyst constricted tubular space.

In an alternative embodiment of the present example, further elements may include a power supply for the first UV radiation source and the second UV radiation source; at least one removable panel on the system; and at least one safety interlock switch. The at least one safety interlock switch may detect removal of the at least one removable panel. The at least one safety interlock switch may switch off power to the power supply for the first UV radiation source and the second UV radiation source when removal of the panel is detected.

### THIRD EXAMPLE

In an example method of purifying air steps may include using a motor driven fan wherein the motor has at least two speeds; using an indoor air handling system with the motor driven fan and with a baffle that has at least an open and a closed position; installing a bypass for air to flow through a constricted space around the baffle, so that air can flow past the baffle when the baffle is in the closed position and through the bypass and back into the air handling system; and disinfecting air in the bypass using a UV lamp and a photocatalyst.

In an alternative embodiment of the present example the bypass may contain a first UV lamp in a confined area of a mirrored cylindrical tube having an inner surface which is exposed to radiation from the UV lamp, and which inner surface is coated with a photocatalyst. The bypass may contain a second UV lamp in a confined area of a photocatalyst cylindrical tube having an inner surface which is exposed to radiation from the UV lamp, which inner surface is a polished mirror surface and reflects the radiation from the UV lamp.

In an alternative embodiment of the present example the air may flow through the mirrored cylindrical tube first and then flow into the photocatalyst cylindrical tube. The air may also flow through the photocatalyst cylindrical tube first, and then flow into the mirrored cylindrical tube. The air may also flow in parallel through the mirrored cylindrical tube and the photocatalyst cylindrical tube at the same time.

### BRIEF DESCRIPTION OF THE DRAWINGS

FIG. 1 is a top down view of an example embodiment of the present invention showing a closed baffle.

FIG. 2 is a top down view of an example embodiment of the present invention showing an open baffle.

FIG. 3 is a top down view of an example embodiment of the present invention showing a first and second fan.

FIG. 4 is a top down view of an example embodiment of the present invention.

FIG. 5 is a side view of an example embodiment of the present invention showing an example of the bypass system.

FIG. 6 is a side view of an example embodiment of the present invention showing an example of the bypass system.

FIG. 7 is a perspective view of example embodiment of the present invention showing tubes.

FIG. 8 is a top down view of and example embodiment of a mirrored tube.

FIG. 9 is a cross sectional view along the line 9-9 of FIG. 8.

FIG. 10 is a top down view of and example embodiment of a photocatalyst tube.

FIG. 11 is a cross sectional view along the line 11-11 of FIG. 10.

FIG. 12 is a top down view of and example embodiment of a mirrored tube.

FIG. 13 is a cross sectional view along the line 13-13 of FIG. 12.

FIG. 14 is a top down view of and example embodiment of a photocatalyst tube.

FIG. 15 is a cross sectional view along the line 15-15 of FIG. 14.

FIG. 16 is a diagram of an example air handling and air purification system.

FIG. 17 is a side view of an alternative example air purification system.

FIG. 18 is a diagram of an example air handling system.

FIG. 19 is a diagram of an example air handling system.

### DETAILED DESCRIPTION

The present invention provides a method of purifying air used in a home or office, or other inhabited space, and a system for purifying air. The embodiments described throughout the specification, including the drawings are example embodiments, and are not intended to limit the scope of the invention.

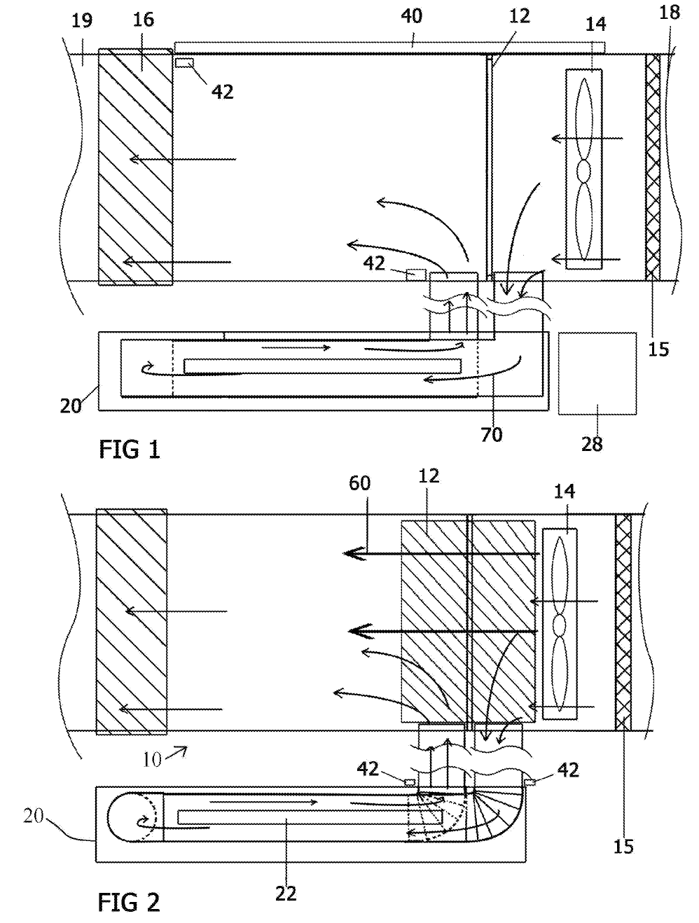

FIGS. 1-4 shows a top down view of part of example air handling systems. The air in the air handling system shown may flow through the heat exchanger path 60 shown in FIG. 2. The air may also flow through an air purification path 70 shown in FIG. 1. A baffle 12 which has an open and a closed position is shown. When the baffle is in the open position, air may flow through a conventional path as with normal heating and cooling systems. When the baffle is closed air may flow through a path which goes through an air purification system at a slow flow rate.

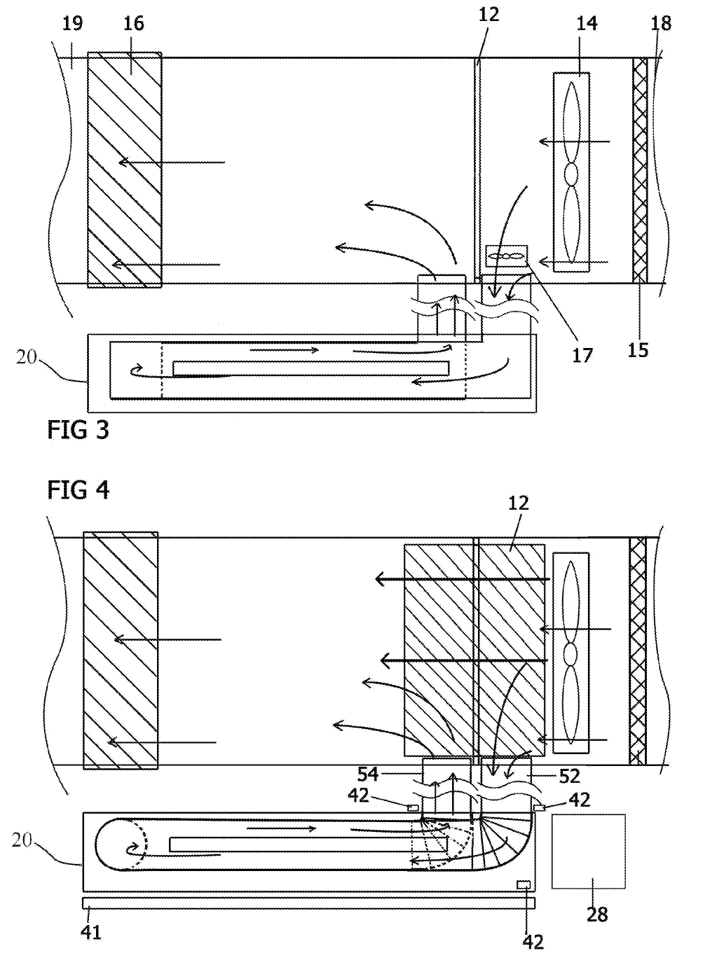

FIG. 1 shows a top down view of an example air handling unit 10. Air may be moved using a motor driven fan 14, and may enter the air handling unit through a supply air duct 18. An air handling unit may be supplied with a motor driven fan that is capable of operating at more than one speed, such as a two speed motor. In the alternative, an air handling unit may have more than one motor, with one motor operating at a normal speed and another motor operating at a slower speed, being a low sped fan as shown in FIG. 3.

An example air handling system may have an air filter unit 15 which may be a simple particulate filter, and which also may include a very fine particulate filter which can remove particles down to 0.1 microns, for example a CleanEffects™ Trane Air filtration system. Other filters and air filtration systems may be used to remove particles from the supply air in the example air handling system, which may remove particles of different sizes. A filter is useful to remove particles as particles may, over time reduce the efficiency of an example air handling system, and the ability of the system to purify air.

An example air handling system may also include a baffle 12 which may have a closed position as shown in FIG. 1, and an open position as shown in FIG. 2. When the baffle is in the closed position, the air may flow at a low rate using a slow speed setting of a fan 14 or a slow speed fan 17. The air may be forced by a fan 14 which is relatively near the baffle and near the air purification path 70 as it appears from the drawings. The drawings, however, are for illustration purposes only, and a fan 14 may be placed at any part of a system which will enable normal use of an air handling system with a heat exchanger 16 (heating or cooling). The air purification path 70 is also placed in the drawings for illustration only, the purification path 70 may be placed at any point in the system.

The purification path 70, may be through a bypass system 20 as shown in FIGS. 1-7 and 16. The bypass system may include a bypass intake 24, and a bypass output 26. In the example illustrations the bypass intake 24, and the bypass output 26 is communicatively attached to the air handling unit 10. The bypass may be attached directly, for example as shown in FIG. 1, and alternatively, the bypass may be communicatively attached through bypass conduits 52 and 54 which allow air to flow from the air handling unit 10 through the bypass 20, and back into the air handling unit 10. For simplicity in illustration the bypass intake 24, and the bypass output 26 are placed close together. The bypass intake 24 and the bypass output 26 may, however, be placed further apart. In the example embodiment discussed, a baffle 12 may be used to direct air flow through the purification path 70, the bypass intake 24 may be placed on one side of the baffle 12, and the bypass output 26 may be placed on the other side of the baffle 12, so that air will be directed through the bypass 20 when the baffle 12 is closed. After passing through the bypass 20 the air may be forced through the air handling system 10 through the return air duct 19, and back into the inhabited space 45 (or inhabited area). The baffle 12 may be moved from a closed to an open position, or vice versa by a change in air pressure. The baffle 12 may be moved from a closed position to an open position and vice versa by operation of a motor.

The bypass may be used without a baffle. For example a bypass may have a separate low speed fan 17 for directing air though the air bypass 20, and a method of directing air so that a substantial portion of the air which passed through the bypass can flow back to the inhabited space. This may be done by directing the air with bypass conduits 52 and 54, with the bypass output conduit 54 being far enough from the input conduit 52, and directed so that air will flow away from the input conduit and back to the inhabited space through the air handling system. Alternatively, the output conduit 52 may be connected to return air directly to the inhabited space.

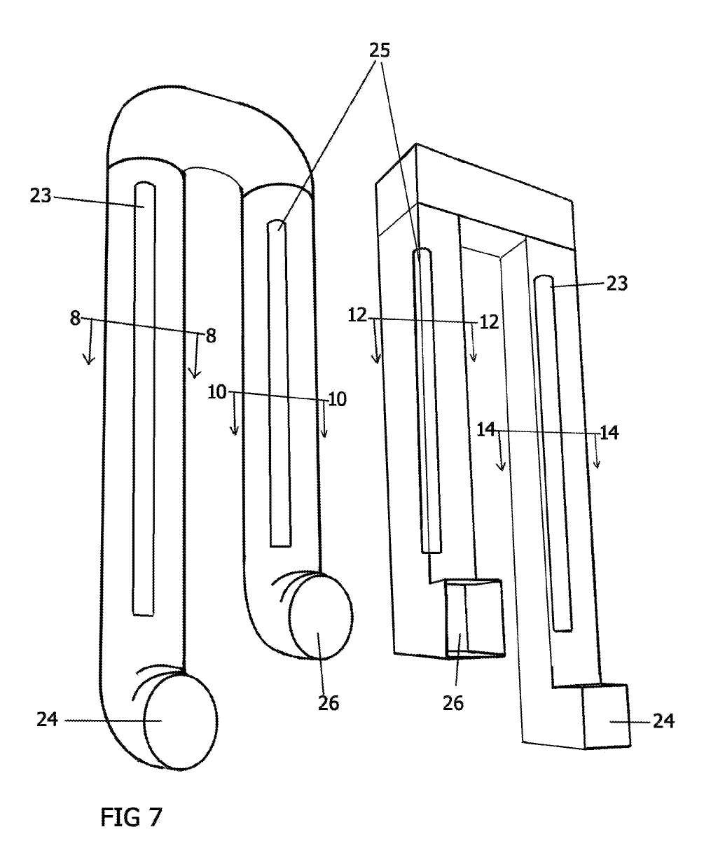

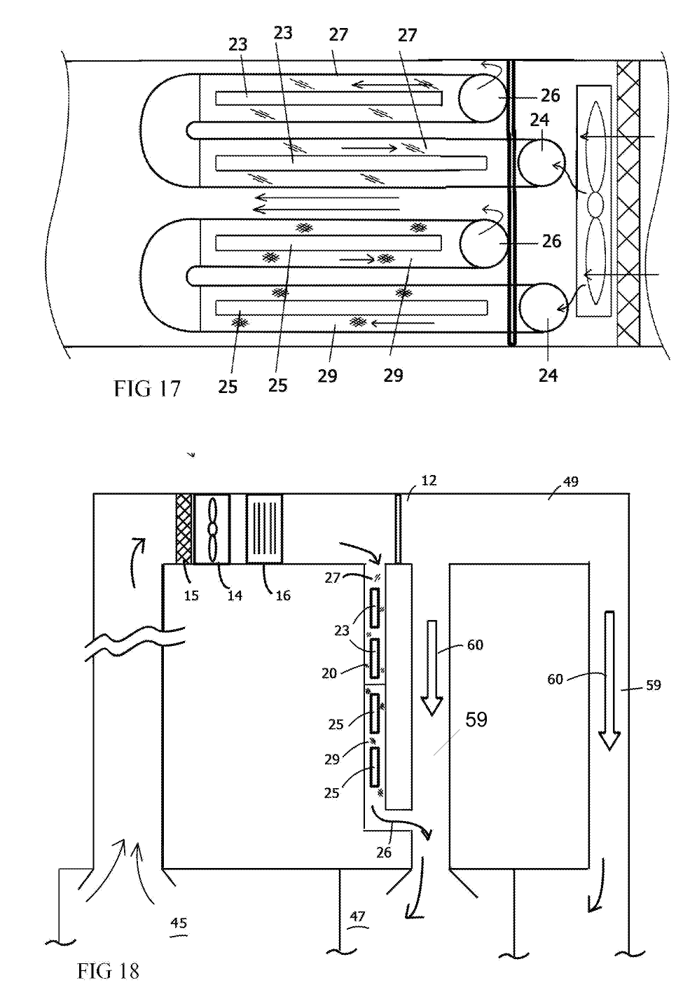

As shown in the example embodiment of FIG. 7 a bypass may contain more than one purification path. Each path may have an independent bypass intake 24 and bypass output 26. Each path may have a first radiation source 25, and a second radiation source 23. More than one air purification path may be configured similar to other air purification paths. Alternatively, each path may have a different type of air purification system. One path may have a DUV purification system, and a different path may have a UV photocatalyst purification system. The shapes and sizes of the different paths may be different, or may be similar. An example bypass system is shown in FIG. 17 where there is more than one path, one path may have first radiation sources 23 with two or more radiation sources, and two or more constricted mirrored tubular spaces 27. In parallel to the path with the first radiation sources may be a path with second radiation sources 25 with two or more radiation sources. The path with the second radiation sources may have two or more constricted photocatalyst tubular spaces 29.

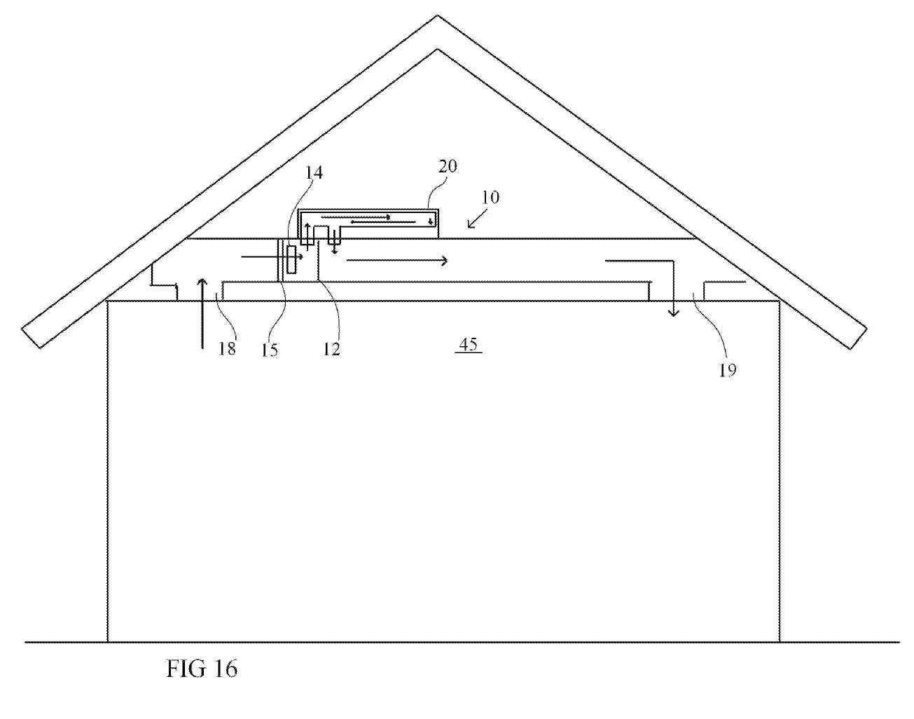

As shown in FIG. 16 an example air purification system may be utilized with a separate path for purifying air. The air purification system may be connected to the air handling system in a home or office (inhabited space 45) so that purified air may be circulated throughout the inhabited space 45.

The air may be forced using a motor driven fan 14 with two different speed settings. The fan 14 may be the main fan for a heating or cooling system, and may force air past a heat exchanger in a heat exchanger path 60 in response to a signal from a thermostat. The fan 14 may produce a flow rate of air through the heat exchanger path which may be at a normal rate for heating or cooling air. When the thermostat no longer indicates that the heating or cooling is needed, the fan 14 may operate at a different speed which is lower.

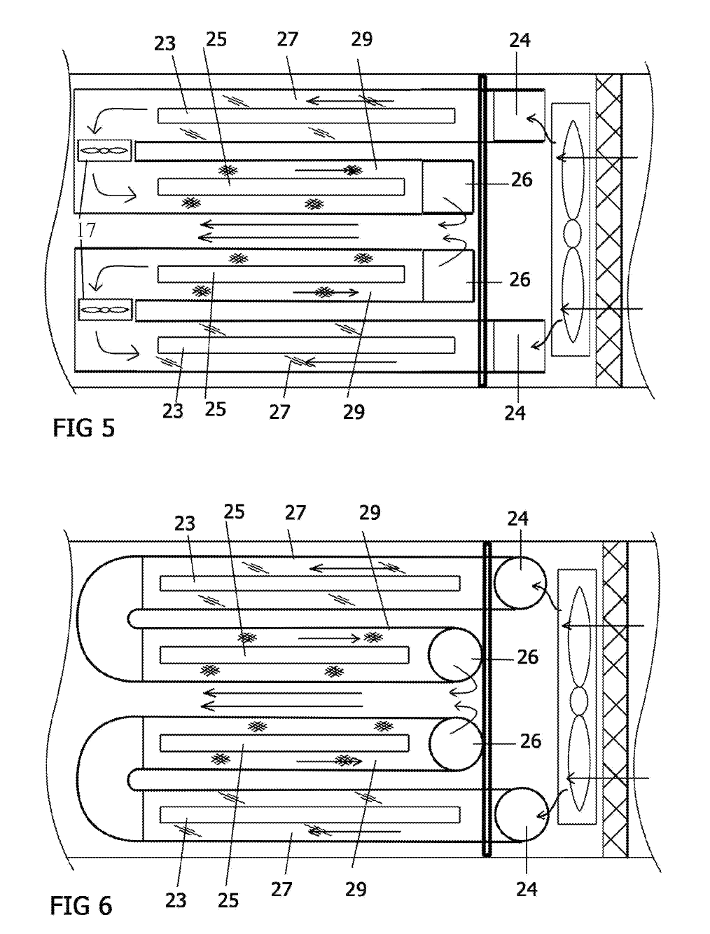

Alternatively, a fan 14 may be configured for a single speed and may be used to force air along the heat exchanger path 60. One or more low speed fans 17 may be used to produce a low flow rate of air through the bypass 20. The low speed fans 17 may be placed at any point along the air purification path 70. For example FIG. 3 shows a low speed fan 17 placed at the bypass intake 24. The low speed fan 17 may also be placed at the bypass output 26, or in the bypass conduits 52 and 54, or inside the bypass as shown in FIG. 5.

The low flow rate of the air through the bypass may be useful to allow air to dwell in the presence of the UV radiation long enough to disinfect and sterilize or kill viruses, bacteria, mold or other desirable substances or chemical compounds in the air. If a separate low speed fan 17 is used, this fan may operate continuously regardless of the operation of a high speed fan for the heating or cooling. Alternatively, the low speed fan 17 may operate in response to a signal from a thermostat, and only operate when a high speed fan is not in operation.

The low flow rate of air through the air purification path 70 may be done continuously by running a fan continuously, or it may be done in intervals, or in response to a signal or switch. One advantage to a continuous circulation of air through an inhabited space 45 may be that the temperature in the inhabited space 45 is more evenly regulated so that there are less areas of varying temperature. According to the present example, air may flow continuously through an air purification path 70 to continuously circulate air from an inhabited space 45, and continuously purify air in the inhabited space 45.

When a fan with two speeds is used, and the fan is operating at a normal rate of speed, air may flow primarily through the heat exchanger path 60, and may also flow through the air purification path 70.

A bypass system 20 may be a system which allows air to enter a separate path which is primarily for purification of the air 70. Air may travel through a different path than the air purification path 70, so that not all of the air that is forced through an air handling unit 10 pass through the air purification unit. Substantially all air which flows through the air purification path 70, however, may be purified. The purification may take place using many different purification systems which are know to reduce or eliminate odors, harmful vapors, viruses and mold. The bypass system may purify, or disinfect the air in the bypass system.

An example purification system is shown in FIGS. 5-7. Air may enter the air purification system through an intake, which may be a bypass intake 24, and exit the air purification system through an output, which may be a bypass output.

An example purification system may be embodied in a bypass 20, which may contain a first radiation source 23, and may also contain a second radiation source 25. The radiation source may be an Ultra Violet (UV) radiation source, and may be one or more UV lamps. The first radiation source 23 may be a UV lamp, and the second radiation source 25 may be a UV lamp of a different wavelength. The first radiation source 23 may be a UV lamp which may be a Deep Ultraviolet (DUV) radiation source. The first radiation source 23 may be selected as a radiation source which can kill viruses. The enclosure where the first radiation source 23 is place may be a mirrored constricted space 27. The enclosure may have a polished surface on the inside of the enclosure so that radiation from the first radiation source 23 may be reflected. The mirrored constricted space may be constricted so that air passing through the space is required to pass within a certain distance from the radiation source. The mirrored surface may reflection the radiation so that air passing through the constricted space will receive radiation from the radiation source, and from the reflection of the radiation source. This may increase the ability of the radiation to interact with viruses and other unwanted biological substances in the air.

UV radiation source may be UV lamps, for example black lamps and Deep Ultraviolet (DUV) or other UV lamps. UV radiation source may also be other sources, for example UV or DUV Light emitting diodes (LEDs).

In an example purification system, the second radiation source 25 may also be a UV lamp. The second radiation source may be a black light, or other UV radiation source. The enclosure where the second radiation source 25 is placed may be a photocatalyst constricted space 29. A photocatalyst, for example titanium dioxide, may be used in the presence of the radiation source 25 to further sterilize mold, and to break down organic compounds. The photocatalyst may be placed in the presence of the radiation source in many different ways known in the arts. The photocatalyst may also be coated on an inside surface of the photocatalyst constricted space 29. The photocatalyst constricted space 29 may be constricted so that air passing through the constricted space may pass within a certain distance of the photocatalyst, or within a certain distance of the radiation source 29.

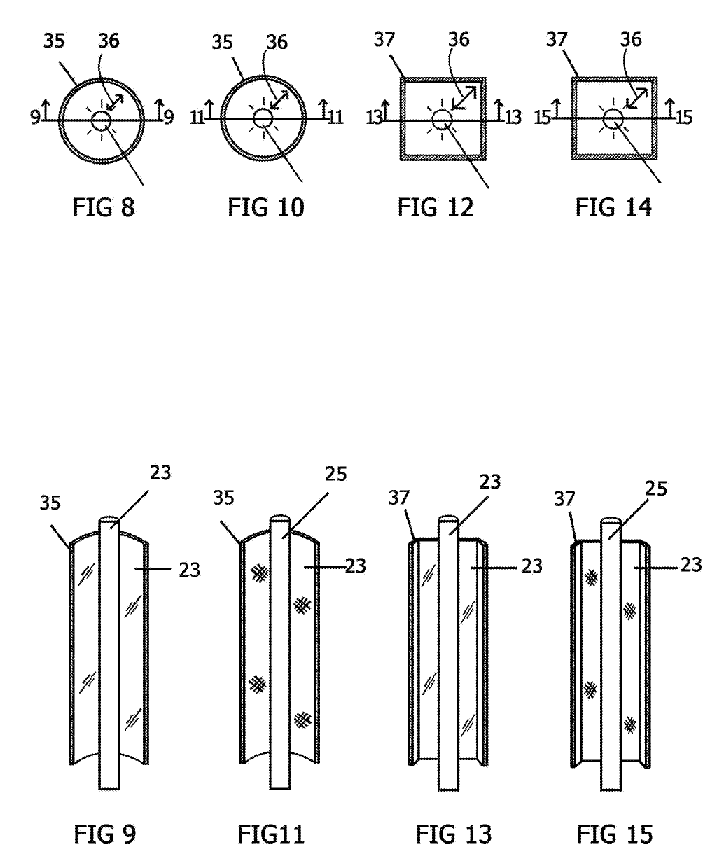

As shown in FIGS. 8-15, a radiation source may be placed in a constricted space which is shaped in the shape of a tube. The tube may be round, or square as shown. The tube may be other shapes or sizes. The constricted space may be formed of a cylindrical tube 35 or of a square tube 37. The distance from the radiation source to the furthest point from the radiation source where air may pass may be a defined distance 36. There may be an ideal distance from the radiation source for the viruses and other compounds in the air to receive a sufficient dose of the radiation so that the viruses and other substances may be killed or sterilized. Radiation emitted from a lamp or many other radiation sources is know to reduce have a reduction in intensity as a function of the distance from radiation source. The ideal maximum distance from the radiation source may depend on the intensity of the radiation from the radiation source. The maximum distance from the radiation source where air may pass may be a distance 36, which may be 6 inches or less, and may be 3 inches or less. A larger distance, for example 12 or 18 inches from the radiation source may also be used.

A filter may be useful to extend the life and increase the effectiveness of the system because particles may become attached to the radiation source, such as a UV lamp, and reduce the amount of radiation, and therefore the dose of radiation received by air passing the radiation source. If radiation source is a UV lamp, then it will need to be changed periodically, and it may be desirable to have the air passing the radiation source to receive a sufficient dose to purify the air shortly before a scheduled lamp change. If a UV lamp becomes coated with particles, then the dose of radiation may decrease to a level where some harmful substances passing by the radiation source will not be neutralized or killed. If a radiation source is a LED UV radiation source, then it may be necessary to clean the source at regular intervals. If a particulate filter is used this may extend the intervals between scheduled maintenance.

Another factor in the amount of minimum radiation received by a virus or other compound in air as it passes through a purification system may be the time that the particle remains in the presence of the radiation source. As the virus or other compound is carried by the air passing through a purification system, the speed, or flow rate of the air, will be a factor in determining the dose of radiation received. The example purification systems shown may control the flow rate of the air, and constrict air passing through a constricted space where a radiation source is present so that all of the air, and particles and substances in the air, may receive a minimum dose of radiation from the radiation source. A minimum dose to kill or sterilize a virus, bacteria, or mold, may be determined and from this the minimum dwell time needed in the presence of a selected radiation source. The size and length of the constricted space, as well as the selection of the flow rate, and radiation source to produce a purification system with a high efficiency in purifying air.

An example purification system may have a constricted space with a radiation source present, and the constricted space may allow the air to pass within about 6 inches of the radiation source. Another example purification system may be configured with a constricted space with a radiation source present, and the constricted space may allow the air to pass within about 3 inches of the radiation source.

It is desirable for a person to avoid direct exposure to from some radiation sources, as they may have harmful effects upon direct exposure. An example embodiment of a purification system using one or more radiation source may include a safety interlock system. An example safety interlock system may include interlock switches 42 which are designed to detect the movement or removal of a panel or other cover. For example, a safety interlock switch 42 may detect the removal of an access panel 40 or 41. The radiation source may be powered by a power supply 28, which may be a ballast, or may be another type of power supply. The safety interlock switch may send a signal to shut off the power supply 28 to the radiation source when the safety interlock switch detects removal of the panel or other cover 40 or 41.

An example purification system may be a bypass 20, which may include a power supply. The bypass may include UV lamps and ballast which need periodic replacement for an efficient system. The bypass may be a complete unit which may be removed as a whole, and replaced as a whole. The bypass may also be a unit with discrete parts which may be removed and replaced. The UV lamps may be mounted in tubes which are coated with a mirror or catalyst coating, and the tubes may be removed with the lamps mounted in them. It may be useful for the lamps and the tubes to be replaced together so that the tubes and lamps may be inspected periodically, and for ease of installing new lamps. The entire bypass unit may also be removed as a whole so that the entire system may be inspected including the ballast, and replaced with ease periodically.

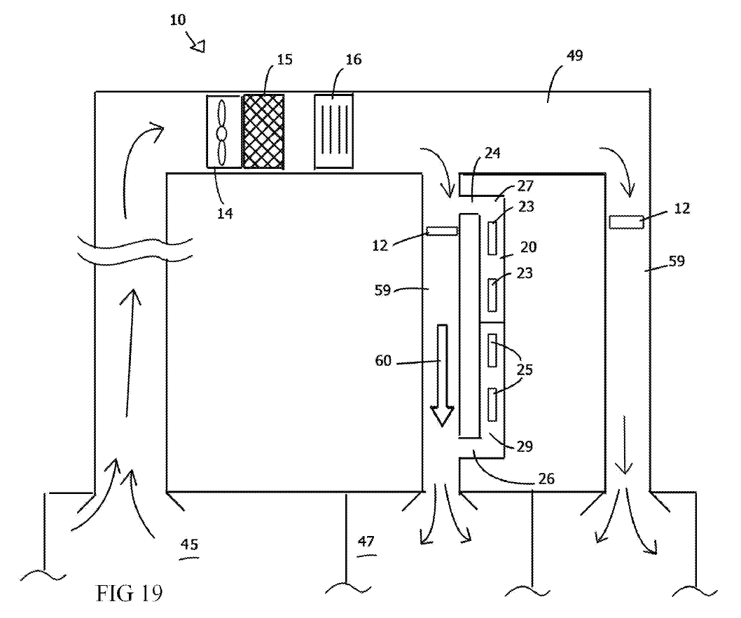

Variations of example systems and methods are shown in FIGS. 18 and 19. Air from inhabited space 45 may be drawn in an air handling system 10 and through a filter 15, using a fan 14. The air may pass a heat exchanger 16 to heat or cool the air. It is not necessary in the example system to humidify the air. A baffle 12 may be in the path of the air flow. Air may flow past the baffle 12 during normal operation of the air handling system 10 and through a main duct 49 into branch ducts 59. The path may be described as a heat exchanger path 60. A bypass 20 may be installed along one of the branch ducts 59. The baffle may operate so that it is closed when air the handling system 10 is not being used to heat or cool the air, and air may flow primarily through the bypass 20. The bypass may be a constricted space and have one or more radiation source 23 and 25. The inner surface of the bypass may be a coated mirror surface 27, or a coated photocatalyst surface 29.

In FIG. 18 one baffle 12 is used and the bypass 20 receives air from the main duct 49, and delivers purified air into a branch duct 59.

In FIG. 19 two or more baffles 12 are used, with one for each branch duct 59, so that when the baffles 12 are closed air will flow primarily through the bypass purification path 20. The bypass 20 receives air from a branch duct 59 and returns air to the branch duct 59.

In the examples of FIGS. 18 and 19 purified air from the bypass is not delivered to all duct branches 59 of the air handling system 10, but is only delivered to one duct branch 59. The inhabited space 45 may have a separate space 47. The separate space 47 may have air which is effectively cleaner than other parts of the inhabited space 45. Air may circulate throughout the inhabited space 45, but the purified air from the bypass 20 may be delivered to one or more separate spaces 47.

The embodiments discussed above are examples to illustrate different aspects of the invention and are not intended to limit the scope of the invention. Other configurations and methods not expressly illustrated or discussed may be implied by the illustrations and examples provided, the scope of the claims are intended to include all such configurations and methods, and other configurations and methods which are made obvious in light of the disclosures above.

1. A method of purifying air comprising:
a) forcing air from within an inhabited space into an air handling system using at least one motor driven fan;
b) filtering air using a particulate filter;
c) exposing the forced air to a first UV radiation source and to a second UV radiation source; the first UV radiation source being in a mirrored constricted tubular space with the first UV radiation source in the constricted tubular space, the mirrored constricted tubular space having a mirrored surface on an inner wall of the tubular space, the mirrored surface being capable of reflecting ultraviolet radiation, the mirrored constricted tubular space constricting the forced air so that the forced air passes within about 3 inches or less from the first UV radiation source; the second UV radiation source being a black light and being in a photocatalyst constricted tubular space with the second UV radiation source in the photocatalyst constricted tubular space, the second tubular space having a coating of titanium dioxide on a surface of an inner wall of the photocatalyst constricted tubular space, the photocatalyst constricted tubular space constricting the forced air so that the forced air passes within about 3 inches or less from the second UV radiation source;
d) returning the air to the inhabited space.

2. The method of claim 1 wherein the forced air flows first through the mirrored constricted tubular space, and then through the photocatalyst constricted tubular space.

3. The method of claim 1 wherein the forced air flows first through the photocatalyst constricted tubular space and then through the mirrored constricted tubular space.

4. The method of claim 1 wherein the forced air flows in parallel through the mirrored constricted tubular space and the photocatalyst constricted tubular space.

5. A method of purifying air according to claim 1 wherein the mirrored constricted tubular space is a mirrored round cylinder and wherein the photocatalyst constricted tubular space is a second round cylinder

6. A method of purifying air according to claim 1 wherein the mirrored constricted tubular space is a mirrored square tube and wherein the photocatalyst constricted tubular space is a second square tube.

7. A method according to claim 1 wherein the step of forcing air from within an inhabited space into an air handling system using at least one motor driven fan, includes forcing air at a normal flow rate for heating or cooling air along a first path, and forcing air at a slow flow rate along a second path.

8. A system for purifying air comprising:
a) a motor driven fan;
b) a particulate filter;
c) a first UV radiation source and a second UV radiation source;
the first UV radiation source being in a mirrored constricted tubular space with the first UV radiation source approximately in the center of the mirrored constricted tubular space, the mirrored constricted tubular space having a mirrored surface on an inner wall of the tubular space, the mirrored surface being capable of reflecting ultraviolet radiation, the mirrored constricted tubular space constricting the forced air so that the forced air passes within about 6 inches or less from the first UV radiation source;
the second UV radiation source being a black light and being in a photocatalyst constricted tubular space with the second UV radiation source approximately in the center of the photocatalyst constricted tubular space, the second tubular space having a coating of titanium dioxide on a surface of an inner wall of the photocatalyst constricted tubular space, the
photocatalyst constricted tubular space constricting the forced air so that the forced air passes within 6 inches or less from the second UV radiation source; wherein the motor driven fan causes air from an inhabited space to be circulated first through a particulate filter, and then through the mirrored constricted tubular space, and through the photocatalyst constricted tubular space, and wherein the motor driven fan causes the air to be driven through a heating or cooling system and returned to the inhabited space.

9. The system for purifying air according to claim 8 wherein the motor driven fan is configured to operate in at least a low speed and a high speed, and wherein the heating or cooling system has a baffle which is open when the motor driven fan is operating at a high speed, and is closed when the motor driven fan is operating at a low speed, and wherein the mirrored constricted tubular space and the photocatalyst constricted tubular space are connected to bypass the baffle so that when the baffle is closed, air will flow primarily through the mirrored constricted tubular space and the photocatalyst constricted tubular space at a low speed; and when the baffle is open the air will flow past the open baffle, and through the mirrored constricted tubular space and the photocatalyst constricted tubular space.

10. The system for purifying air according to claim 9 wherein the air flows first through the mirrored constricted tubular space and then through the photocatalyst constricted tubular space.

11. The system for purifying air according to claim 9 wherein the air flows in parallel through the mirrored constricted tubular space and the photocatalyst constricted tubular space.

12. The system for purifying air according to claim 8 further comprising: a power supply for the first UV radiation source and the second UV radiation source; at least one removable panel on the system; and at least one safety interlock switch; wherein the at least one safety interlock switch detects removal of the at least one removable panel, and wherein the at least one safety interlock switch switches off power to the power supply for the first UV radiation source and the second UV radiation source when removal of the panel is detected.

13. A method of purifying air comprising:
Using a motor driven fan wherein the motor has at least two speeds;
Using an indoor air handling system with the motor driven fan and with a baffle that has at least
an open and a closed position;
Installing a bypass for air to flow through a constricted space around the baffle, so that air can flow past the baffle when the baffle is in the closed position and through the bypass and back into the air handling system;
Disinfecting air in the bypass.

14. The method of purifying air according to claim 13 wherein the disinfecting is performed using a UV lamp and a photocatalyst.

15. The method of purifying air according to claim 14 wherein the bypass contains at least a first UV lamp in a confined area of a mirrored cylindrical tube having an inner surface which is exposed to radiation from the UV lamp, and which inner surface is coated with a photocatalyst;
and wherein the bypass contains a second UV lamp in a confined area of a photocatalyst cylindrical tube having an inner surface which is exposed to radiation from the UV lamp, which inner surface is a polished mirror surface and reflects the radiation from the UV lamp.

16. The method of purifying air according to claim 14 wherein the air flows through the mirrored cylindrical tube first and then flows into the photocatalyst cylindrical tube.

17. The method of purifying air according to claim 14 wherein the air flows through the photocatalyst cylindrical tube first, and then flows into the mirrored cylindrical tube.

18. The method of purifying air according to claim 14 wherein the air flows in parallel through the mirrored cylindrical tube and the photocatalyst cylindrical tube at the same time.
